(19) United States
(12) Patent Application Publication
(10) Pub. No.: US 2024/0386993 A1
(43) Pub. Date: Nov. 21, 2024

(54) Binding Analysis Of Protein Molecule And Ligand Molecule

(71) Applicant: Microsoft Technology Licensing, LLC, Redmond, WA (US)

(72) Inventors: Tong WANG, Beijing (CN); Bin SHAO, Beijing (CN); Tie-Yan LIU, Beijing (CN)

(21) Appl. No.: 18/687,404

(22) PCT Filed: Jul. 25, 2022
(86) PCT No.: PCT/US2022/038111

§ 371 Date: Feb. 28, 2024

(30) Foreign Application Priority Data
Aug. 31, 2021 (CN) 202111013709.1

(51) Int. Cl. G16B 15/30; G16B 40/20

(52) U.S. Cl. CPC G16B 15/30; G16B 40/20

(57) ABSTRACT

According to implementations of the subject matter described herein, a solution for molecular binding analysis is provided. In the solution, a first feature representation determined based on a structure of a ligand molecule may be obtained, and a second feature representation determined based on a structure of a protein molecule may be obtained. A third feature representation of a complex structure may be determined, wherein the complex structure is built based on the protein molecule and the ligand molecule. The first feature representation, the second feature representation and the third feature representation may be used to generate an aggregate feature representation so as to determine evaluation information on the binding between the ligand molecule and the protein molecule. The evaluation information may indicate the effectiveness of the binding or indicate the affinity of a binding pose of the binding. Thereby, more efficient and accurate binding analysis can be realized.

### BACKGROUND

An important task in drug discovery is to analyze whether protein molecules (e.g., target proteins) and small drug molecules (e.g., also referred to as ligand molecules) can be bound effectively. The traditional drug discovery process relies on chemical experiments to study intermolecular binding.

In recent years, with the development of computer technology, some machine learning techniques have gradually been applied to predict the binding between protein molecules and ligand molecules. Predicting intermolecular binding through machine learning techniques can greatly reduce the cost of drug discovery, and people also pay more and more attention to how to improve the accuracy of molecular set prediction based on machine learning techniques.

### SUMMARY

According to implementations of the subject matter described herein, a solution for molecular binding analysis is provided. In the analysis solution, a first feature representation determined based on a structure of a ligand molecule may be obtained, and a second feature representation determined based on a structure of a protein molecule may be obtained. Further, a third feature representation of a complex structure may be determined, wherein the complex structure is built based on the protein molecule and the ligand molecule. Further, the first feature representation, the second feature representation and the third feature representation may be used to generate an aggregate feature representation so as to determine evaluation information on the binding between the ligand molecule and the protein molecule. The evaluation information may indicate the effectiveness of the binding or indicate the affinity of a binding pose of the binding. Thereby, more efficient and accurate binding analysis can be realized.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a block diagram of an example computing device according to some implementations of the subject matter described herein;

FIG. 2 illustrates a flowchart of the process of molecular binding analysis according to some implementations of the subject matter described herein;

FIG. 3 illustrates a schematic block diagram of a molecular binding analysis module according to some implementations of the subject matter described herein;

FIG. 4 illustrates a schematic view of an attention model according to some implementations of the subject matter described herein;

FIG. 5 illustrates a schematic view of the comparison between a binding effectiveness prediction solution according to some implementations of the subject matter described herein and other solutions; and

FIG. 6 illustrates a schematic view of the comparison between a binding pose prediction solution according to some implementations of the subject matter described herein and other solutions.

Throughout the drawings, the same or similar reference signs refer to the same or similar elements.

### DETAILED DESCRIPTION

The subject matter described herein will now be discussed with reference to several example implementations. It is to be understood these implementations are discussed only for the purpose of enabling persons skilled in the art to better understand and thus implement the subject matter described herein, rather than suggesting any limitations on the scope of the subject matter.

As used herein, the term “includes” and its variants are to be read as open terms that mean “includes, but is not limited to.” The term “based on” is to be read as “based at least in part on.” The term “one implementation” and “an implementation” are to be read as “at least one implementation.” The term “another implementation” is to be read as “at least one other implementation.” The terms “first,” “second,” and the like may refer to different or same objects. Other definitions, explicit and implicit, may be included below.

As discussed above, the traditional drug discovery process relies on chemical experiments to detect the effectiveness of the binding between proteins and ligand molecules. For a specific target protein, experimenters might have to spend a lot of labor and time costs to screen out small molecules that can be effectively bound to the target protein from mass small molecules.

In recent years, computer aided drug discovery (CADD) has been gradually applied to reduce the drug discovery cost. However, due to the limited accuracy of CADD technology such as machine-based learning technology, people desire to improve the accuracy of molecule binding analysis and thereby aid the drug discovery work.

According to implementations of the subject matter described herein, a solution is provided for molecule binding analysis. In the analysis solution, a first feature representation determined based on a structure of a ligand molecule may be obtained, and a second feature representation determined on a structure of a protein molecule may be obtained. Further, a third feature representation of a complex structure may be obtained, wherein the complex structure is built based on the protein molecule and the ligand molecule. The first feature representation, the second feature representation and the third feature representation may be used to generate an aggregate feature representation to determine evaluation information on the binding of the ligand molecule and the protein molecule. The evaluation information may indicate the effectiveness of the binding or may indicate the affinity of a binding pose of the binding. Thereby, it is possible to realize more effective and accurate binding analysis.

By aggregating features of the ligand molecule, the protein molecule and the complex structure, embodiments of the subject matter described herein can fully consider the intermolecular interactions and further improve the accuracy of binding analysis.

The basic principle and several example implementations of the subject matter described herein will be illustrated with reference to the drawings below.

### Example Device

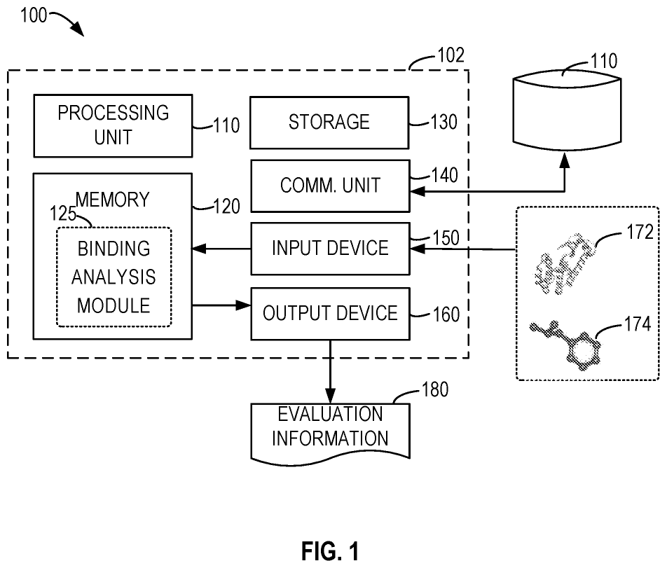

FIG. 1 illustrates a schematic block diagram of an example device 100 that can implement implementations of the subject matter described herein. It should be understood that the device 100 shown in FIG. 1 is only exemplary and shall not constitute any limitation on the functions and scopes of the implementations described by the subject matter described herein. As shown in FIG. 1, components of the device 100 may include, but is not limited to, one or more processors or processing units 110, a memory 120, a storage device 130, one or more communication units 140, one or more input devices 150, and one or more output devices 160.

In some implementations, the device 100 may be implemented as various user terminals or service terminals. The service terminals may be servers, large-scale computing devices, and the like provided by a variety of service providers. The user terminal, for example, is a mobile terminal, a fixed terminal or a portable terminal of any type, including a mobile phone, a site, a unit, a device, a multimedia computer, a multimedia tablet, Internet nodes, a communicator, a desktop computer, a laptop computer, a notebook computer, a netbook computer, a tablet computer, a Personal Communication System (PCS) device, a personal navigation device, a Personal Digital Assistant (PDA), an audio/video player, a digital camera/video, a positioning device, a television receiver, a radio broadcast receiver, an electronic book device, a gaming device or any other combination thereof consisting of accessories and peripherals of these devices or any other combination thereof. It may also be predicted that the device 100 can support any type of user-specific interface (such as a “wearable” circuit, and the like).

The processing unit 110 may be a physical or virtual processor and may execute various processing based on the programs stored in the memory 120. In a multi-processor system, a plurality of processing units executes computer-executable instructions in parallel to enhance parallel processing capability of the device 100. The processing unit 110 can also be known as a central processing unit (CPU), microprocessor, controller and microcontroller.

The device 100 usually includes a plurality of computer storage mediums. Such mediums may be any attainable medium accessible by the device 100, including but not limited to, a volatile and non-volatile medium, a removable and non-removable medium. The memory 120 may be a volatile memory (e g., a register, a cache, a Random Access Memory (RAM)), a non-volatile memory (such as, a Read-Only Memory (ROM), an Electrically Erasable Programmable Read-Only Memory (EEPROM), flash), or any combination thereof. The memory 120 may include one or more encoding/decoding modules 125, which program modules are configured to perform various encoding functions/decoding functions described herein. A binding analysis module 125 module may be accessed and operated by the processing unit 110 to realize corresponding functions. The storage device 130 may be a removable or non-removable medium, and may include a machine-readable medium (e.g., a memory, a flash drive, a magnetic disk) or any other medium, which may be used for storing information and/or data and be accessed within the device 100.

Functions of components of the device 100 may be realized by a single computing cluster or a plurality of computing machines, and these computing machines may communicate through communication connections. Therefore, the device 100 may operate in a networked environment using a logic connection to one or more other servers, a Personal Computer (PC) or a further general network node. The device 100 may also communicate through the communication unit 140 with one or more external devices (not shown) as required, where the external device, e.g., a storage device, a display device, and so on, communicates with one or more devices that enable users to interact with the device 100, or with any device (such as a network card, a modem, and the like) that enable the device 100 to communicate with one or more other computing devices. Such communication may be executed via an Input/Output (I/O) interface (not shown).

The input device 150 may be one or more various input devices, such as a mouse, a keyboard, a trackball, a voice-input device, and the like. The output device 160 may be one or more output devices, e.g., a display, a loudspeaker, a printer, and so on.

In some implementations, as shown in FIG. 1, the device 100 may, for example, receive identifications corresponding to a protein molecule 172 and a ligand molecule 174 through the input device 150. For example, a user may input a PDB file through the input device 150 to indicate the corresponding protein molecule 172. The user may further input a character string in SMILES (simplified molecular input line entry system) format through the input device 150 to indicate the corresponding ligand molecule 174.

In some implementations, the binding analysis module 125 may determine evaluation information 180 on the binding between the protein molecule 172 and the ligand molecule 174 based on a structure of the protein molecule 172 and a structure of the ligand molecule 174, the evaluation information 180, for example, including a prediction result output by the binding analysis module 125. The process for determining the evaluation information 180 will be described in detail below.

### Molecular Binding Analysis

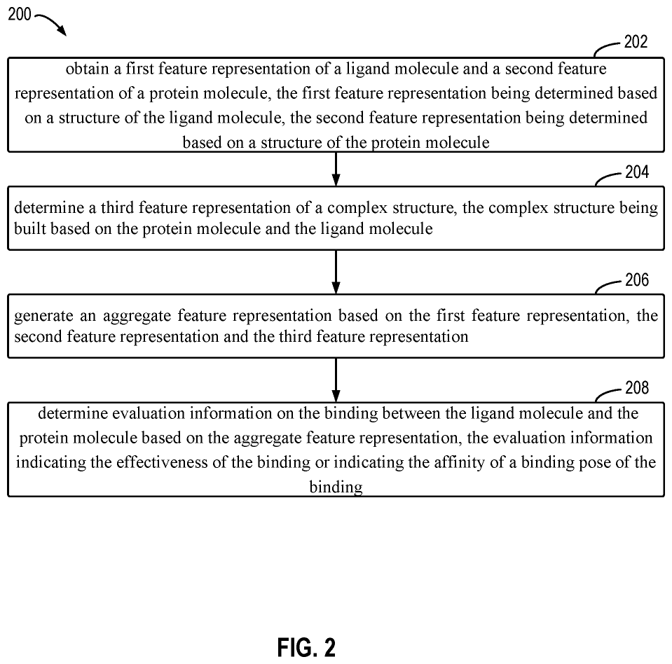

FIG. 2 shows a flowchart of a process 200 of molecular binding analysis according to some implementations of the subject matter described herein. The process 200 may be implemented by the device 110 in FIG. 1, and specifically, may be implemented by the binding analysis module 125 in the device 100.

As shown in FIG. 2, at block 202, the device 100 obtains a first feature representation of the ligand molecule 174 and a second feature representation of the protein molecule 172, wherein the first feature representation is determined based on the structure of the ligand molecule 174, and the second feature representation is determined based on the structure of the protein molecule 172.

In some implementations, the device 100 may determine the structure of the protein molecule 172 based on a user input, so as to generate the second feature representation. The protein molecule 172 may be a target protein to be analyzed in the drug discovery process. The user may, for example, provide to the device 100 a PDB file that corresponds to the to-be-analyzed protein molecule 172.

In some implementations, the device 100 may also determine the structure of the ligand molecule 174 based on a user input. For example, the user may provide to the device 100 information on the protein molecule 172 and the ligand molecule 174 which are desired to be analyzed. As an example, the user may provide a SMILES character string corresponding to the ligand molecule 174, and the device 100 may further determine the structure of the ligand molecule 174 based on the SMILES character string.

In some implementations, the molecular binding analysis module 125 may, for example, be used to screen out a ligand molecule that can be bound with the protein molecule 172 from a group of candidate ligand molecules. In this case, the device 100 may automatically use the candidate ligand molecule in the group of ligand molecules as the to-be-analyzed ligand molecule 174 and further determine the structure information of the ligand molecule 174.

In some implementations, the device 100 may determine a first feature representation based on the structure information of the ligand molecule 174. In some implementations, the first feature representation may comprise a feature graph, which may comprise multiple nodes and edges between the nodes. In some implementations, each node in the feature graph may correspond to an atom in the ligand molecule 174, and an edge may correspond to the binding between atoms.

In some implementations, the first feature representation may comprise node features for characterizing atoms. The node features may be used to characterize, for example, atom symbols (e.g., C, N, O, F, P, Cl, Br, B, H, etc.), the number of covalent bonds, the state of electrical charge, the number of free radical electrons, Hybridization state, aromaticity, the number of connected hydrogens, whether the atom is chiral center, chirality type, amino acid type, or a combination of one or more of the above.

In some implementations, the first feature representation may further comprise edge features for characterizing bonding relationships between atoms. The edge features may be used to characterize, for example, bond type (e.g., single, double, aromatic bond, etc.), conjugation state, whether the bond is in ring, stereo, connection type (e.g., protein-protein, protein-ligand, ligand-ligand), distance information, or a combination of one or more of the above.

The device 100 may determine a second feature representation based on the structure of the protein molecule 172. Similarly, the second feature representation may also comprise a feature graph and comprise node features for characterizing atoms in the protein molecule 172 and edge features for characterizing bonding relationships between atoms. Information characterized by the node features and the edge features may be similar to the above discussed first feature representation.

In some implementations, regarding the second feature representation, the edge features included may further characterize whether a pair of atoms corresponding to an edge are located on the same amino acid.

At block 204, the device 100 may determine a third feature representation of a complex structure, wherein the complex structure is built based on the protein molecule 172 and the ligand molecule 174. Depending on the specific usage of the binding analysis module 125, the complex structure may be determined based on different ways.

In some embodiments, the binding analysis module 125 may be used to analyze whether the protein molecule 172 and the ligand molecule 174 can be bound effectively. In this case, the device 100 may further build a complex structure based on the protein molecule 172 and the ligand molecule 174.

Specifically, the device 100 may first determine a binding pocket of the protein molecule 172. In some implementations, information of the binding pocket of the protein molecule 172 may be determined based on a user input. For example, the user may characterize the binding pocket of the protein molecule 172 through a PDB file.

Further, the device 100 may determine a group of candidate complex structures based on the binding pocket of the protein molecule 172. Specifically, the device 100 may place the ligand molecule 174 into the binding pocket in a proper way, thereby generating a candidate complex structure.

Additionally, the device 100 may determine a target complex structure from the group of candidate complex structures based on binding free energy (BFE) corresponding to the group of candidate complex structures. For example, the device 100 may select a candidate complex structure with the minimum binding free energy from the group of candidate complex structures, as the target complex structure.

In some implementations, the binding analysis module 125 may be used to evaluate the binding pose between the protein molecule 172 and the ligand molecule 174. In this case, the complex structure may be built by the user or determined from a group of candidate complex structures which are automatically built by the device 100.

Further, after determining the complex structure, the device 100 may generate a third feature representation of the complex structure. Similar to the first feature representation and the second feature representation, the third feature representation may also comprise a feature graph and comprise node features for characterizing atoms in the complex structure and edge features for characterizing binding relationships between atoms. Information characterized by the node features and the edge features may be similar to the above discussed first feature representation.

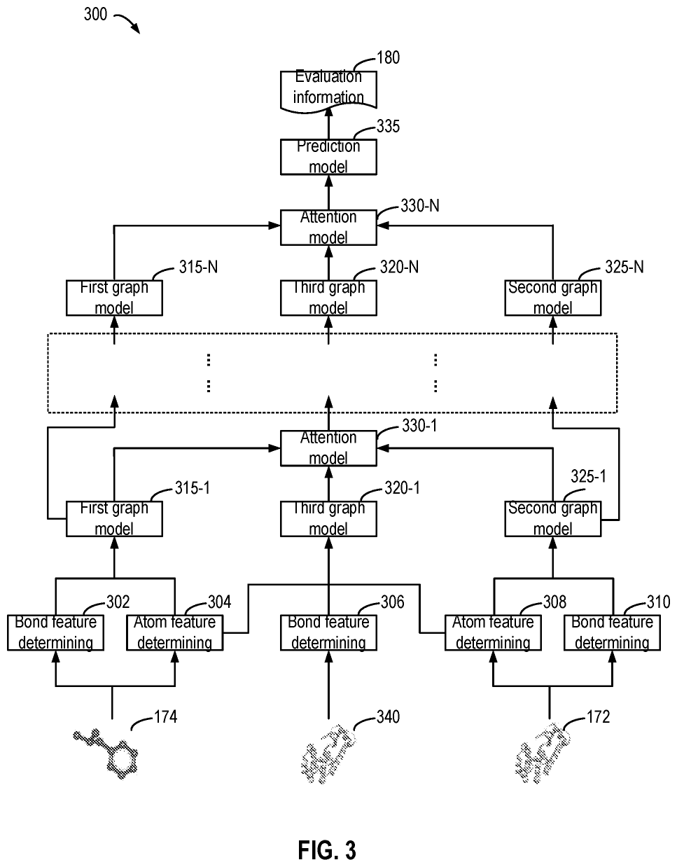

Still with reference to FIG. 2, at block 206, the device 100 generates an aggregate feature representation based on the first feature representation, the second feature representation and the third feature representation. The process for generating the aggregate feature representation will be described in detail with reference to FIG. 3, which shows a schematic block diagram 300 of the molecular binding analysis module 125 according to some implementations of the subject matter described herein.

As shown in FIG. 3, the binding analysis module 125 may use a bond feature determining module 302 to determine edge features of the ligand molecule 174 and use an atom feature determining module 304 to determine node features of the ligand molecule 174, thereby obtaining the first feature representation.

In addition, the binding analysis module 125 may use a bond feature determining module 310 to determine edge features of the protein molecule 172 and use an atom feature determining module 308 to determine node features of the protein molecule 172, thereby obtaining the second feature representation.

As shown in FIG. 3, the binding analysis module 125 may use a bond feature determining module 305 to determine edge features of a complex structure 340 and use a combination of node features of the protein molecule 172 and node features of the ligand molecule 174 as node features of the complex structure 340, thereby obtaining the third feature representation.

In some implementations, as shown in FIG. 3, the binding analysis module 125 may further comprise an attention module 330-1, which is configured to generate an intermediate feature representation based on a group of inputs corresponding to the ligand molecule 174, the complex structure 340 and the protein molecule 172.

In some implementations, the binding analysis module 125 may determine a first input into the attention module 330-1 based on the first feature representation. In some implementations, the binding analysis module 125 may use a first graph model 315-1 to process the first feature representation to generate the first input into the attention model 330-1.

In some implementations, the first graph model 315-1 may comprise a graph transformer model, which is configured to use a multi-head attention mechanism to update the first feature representation based on edge features corresponding to an edge and node features of a pair of nodes corresponding to the edge, in the first feature representation. In this way, the node features and the edge features can be updated based on the binding between nodes.

Similarly, the binding analysis module 125 may use a second graph model 325-1 and a third graph model 320-1 to process the second feature representation and the third feature representation, respectively, so as to determine a second input and a third input into the attention model 330-1.

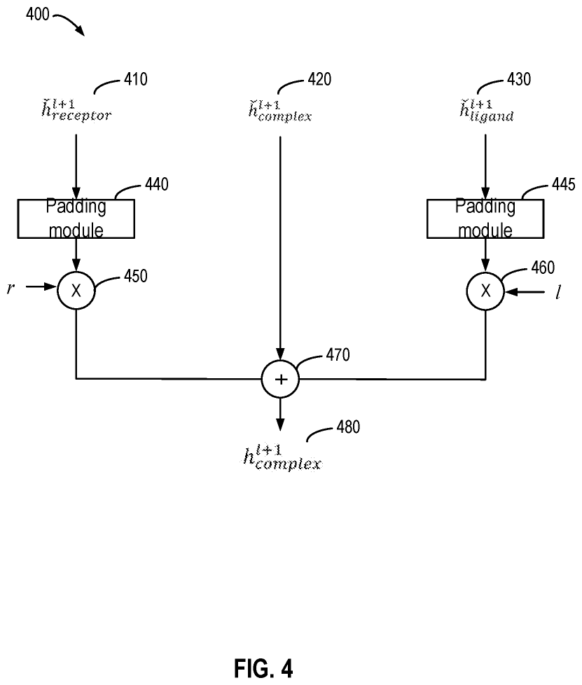

FIG. 4 further shows a schematic view 400 of an attention model according to some implementations of the subject matter described herein. As depicted, a second input 410 may be denoted as h̆_{receptor}^{l+1}, a third input 420 may be denoted as h̆_{complex}^{l+1}, and the first input 410 may be denoted as h̆_{ligand}^{l+1}, wherein l corresponds to the serial number of the attention model 330-1.

As shown in FIG. 4, the attention model 330-1 may use a padding module 440 to process the second input 410, so as to cause the number of feature dimensions to be the same as the number of feature dimensions of the third input 420. For example, the attention model 330-1 may extend the second input 410 to feature dimensions that correspond to the third input 420 through zero padding.

Similarly, the attention model 330-1 may use a padding module 445 to process the first input 430 so as to cause the number of feature dimensions to be the same as the number of feature dimensions of the third input 420.

Further, the attention model 330-1 may use multipliers 450, 460 and an adder 470 to calculate a weighted sum of the padded first input, second input and third input 420, so as to determine an intermediate feature representation 480. For example, the intermediate feature representation 480 may be denoted as:

$${h}_{complex}^{l + 1} = {\breve{h}}_{complex}^{l + 1} + r \times Concat ( [ {0}_{ligand} , {\breve{h}}_{receptor}^{l + 1} ] ) + l \times Concat ( [ {\breve{h}}_{ligand}^{l + 1} , {0}_{receptor} ] ) \qquad ( 1 )$$

wherein concat denotes concatenation operation, and r and l denote weight coefficients.

In some implementations, as shown in FIG. 3, the binding analysis module 125 may comprise multiple layers (also referred to as passing layers) consisting of a first graph model, a second graph model, a third graph model and an attention model. The first graph model in each layer may be configured to receive an output of a corresponding first graph model of the previous layer, the second graph model may be configured to receive an output of a corresponding second graph model of the previous layer, and the third graph model may be configured to receive an output of a corresponding attention model of the previous layer. Further, outputs of the first graph model, the second graph model and the third graph model are used as a group of inputs into the attention model in the current layer, so as to determine a new intermediate feature representation.

In the example of FIG. 3, the binding analysis module 125 may, for example, comprise N layers, wherein the N-th layer comprises a first graph model 315-N, a second graph model 325-N, a third graph model 320-N and an attention model 330-N. The attention model 330-N may have a structure as discussed with reference to FIG. 4 and generate an aggregate feature representation.

Still with reference to FIG. 2, at 208, the device 100 determines evaluation information 180 on the binding between the ligand molecule 174 and the protein molecule 172 based on the aggregate feature representation, wherein the evaluation information 180 indicates the effectiveness of the binding or indicates the affinity of a binding pose of the binding.

In some embodiments, as shown in FIG. 3, the binding analysis module 125 may further comprise a prediction model 335, which is configured to determine the evaluation information 180 based on the aggregate feature representation. That is, the evaluation information 180 is a prediction result output by the prediction model 335.

In some embodiments, parameters of the prediction model 335, the one or more first graph models 315-1 to 315-N, the one or more second graph models 325-1 to 325-N, the one or more third graph models 320-1 to 320-N, and the one or more attention models 330-1 to 330-N are determined through co-training.

In some implementations, the binding analysis module 125 may comprise a first analysis model, which may have a structure as described in FIG. 3 for determining whether the protein molecule 172 and the ligand molecule 174 may be effectively bound.

Specifically, the binding analysis module 125 may obtain a group of candidate ligand molecules, which may be used to determine whether a drug molecule can be effectively bound with the target protein. Further, the binding analysis module 125 may use the first analysis model to process multiple pairs of protein molecule-candidate ligand molecule and determine the effectiveness of the binding of each pair of protein molecule-candidate ligand molecule based on evaluation information generated by the first analysis model.

Further, the binding analysis module 125 may screen out a target ligand molecule which can be effectively bound with the target protein from the group of candidate ligand molecules based on the determined effectiveness of the binding. Thereby, the embodiments of the subject matter described herein can efficiently realize the screening of bindable ligand molecules for specified protein molecules.

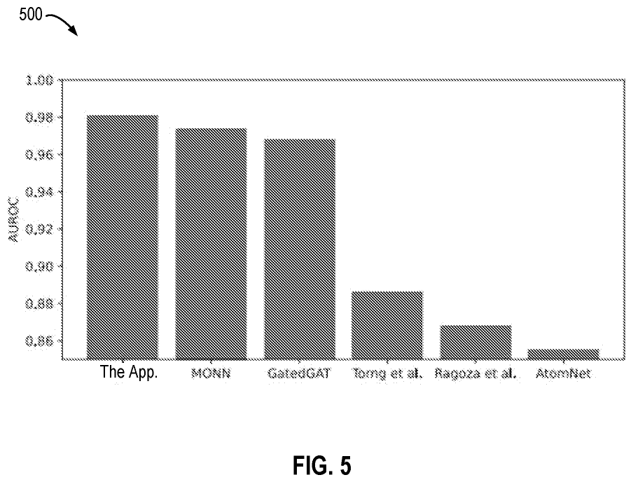

FIG. 5 shows a schematic view 500 of the comparison between the binding effectiveness prediction solution according to some implementations of the subject matter described herein and other solutions. As depicted, when using the solution of the subject matter described herein for effectiveness prediction, the present solution can achieve higher AUROC (area under the receiver operating characteristic curve) than other solutions, i.e., improving the accuracy of the binding effectiveness prediction.

In some implementations, the binding analysis module 125 may comprise a second analysis model, which may have a structure as described in FIG. 3 for determining the affinity of the binding pose of the binding between the protein molecule 172 and the ligand molecule 174 in the complex structure.

Specifically, the binding analysis module 125 may build a group of candidate complex structures based on the protein molecule 172 and the ligand molecule 174, wherein the group of candidate complex structures correspond to a group of candidate binding poses.

Further, the binding analysis module 125 may use the second analysis model to determine evaluation information corresponding to the group of candidate complex structures, so as to determine the affinity of the group of candidate binding poses.

Additionally, the binding analysis module 125 may determine a target binding pose from the group of candidate binding poses based on the affinity indicated by the evaluation information. In some implementations, the evaluation information may indicate two kinds of states (e.g., through two kinds of labels) to indicate whether the candidate binding pose is an effective binding pose. Alternatively, the evaluation information may also be used to indicate a score of the affinity (e.g., by a value between 0 and 1).

As an example, the binding analysis module 125 may screen out a binding pose which is identified as effective or a binding pose whose affinity score is larger than a threshold, from the group of candidate binding poses as the target binding pose. Thereby, the embodiments of the subject matter described herein can efficiently realize the determining of the binding pose between the specified protein molecule and the ligand molecule.

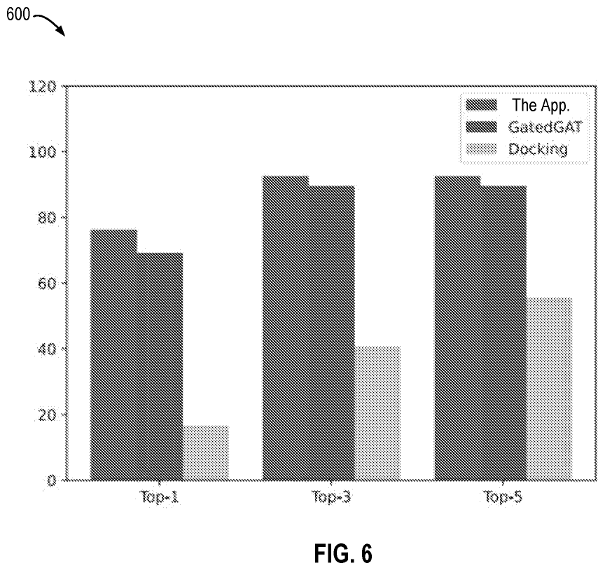

FIG. 6 shows a schematic view of the comparison between the binding pose prediction solution according to some implementations of the subject matter described herein and other solutions. The vertical axis in FIG. 6 represents the percentage of the complex structures that corresponding to the binding poses output by the model and whose RMSD (root-square deviation) is smaller than 2 Å, TOP-1 of the horizontal axis represents the best binding pose, TOP-2 represents the top-two binding poses, and TOP-3 represents the TOP-three binding poses. As seen from FIG. 6, the embodiments of the subject matter described herein can provide more accurate binding pose prediction.

In some embodiments, the binding analysis module 125 may comprise the first analysis model and the second analysis model at the same time. The binding analysis module 125 may use the first analysis model to screen out at least one target ligand molecule from a group of candidate ligand molecules, and use the second analysis model to further determine an effective binding pose between the protein molecule and the at least one target ligand molecule.

It should be understood that the first analysis model and the second analysis model may have similar model structures, whereas the number of passing layers may differ, or dimensions of features used in each model may also differ.

In addition, during the training process, the first analysis model and the second analysis model may use different training data. The first analysis model for determining the effectiveness of the binding may use a training dataset built based on real experimental data. The second analysis model for determining the affinity of the binding pose may use RMSD of the crystal corresponding to the complex structure, for example, the binding pose whose RMDS is smaller than 2 Å may be determined as a positive sample, and the binding pose whose RMSD is larger than 4 Å may be determined as a negative sample.

In this way, the embodiments of the subject matter described herein can effectively screen out the ligand molecule that can be effectively bound with the protein molecule, and provide information on the effective binding pose between the protein molecule and the ligand molecule.

### Example Implementations

Some example implementations of the subject matter described herein are listed below.

In a first aspect, the subject matter described herein provides a method for molecular binding analysis. The method comprises: obtaining a first feature representation of a ligand molecule and a second feature representation of a protein molecule, the first feature representation being determined based on a structure of the ligand molecule, the second feature representation being determined based on a structure of the protein molecule; determining a third feature representation of a complex structure, the complex structure being built based on the protein molecule and the ligand molecule; generating an aggregate feature representation based on the first feature representation, the second feature representation and the third feature representation; and determining evaluation information (may also be referred to as prediction information) on the binding between the ligand molecule and the protein molecule based on the aggregate feature representation, the evaluation information indicating the effectiveness of the binding or indicating the affinity of a binding pose of the binding.

In some implementations, at least one of the first feature representation, the second feature representation and the third feature representation is a feature graph comprising node features and edge features, the node features being used to characterize atoms in a molecule, the edge features being used to characterize binding relationships between atoms.

In some implementations, generating an aggregate feature representation based on the first feature representation, the second feature representation and the third feature representation comprises: determining a first group of inputs based on the first feature representation, the second feature representation and the third feature representation; using an attention model to process the first group of inputs so as to determine a first intermediate feature representation; and determining the aggregate feature representation based on the first intermediate feature representation.

In some implementations, the attention model comprises a first attention model, and determining the aggregate feature representation based on the first intermediate feature representation comprises: determining a second group of inputs based on the first feature representation, the second feature representation and the first intermediate feature representation; using a second attention model to process the second group of inputs so as to determine a second intermediate feature representation; and determining the aggregate feature representation based on the second intermediate feature representation.

In some implementations, a parameter of the attention model is determined based on training a binding analysis model, the binding analysis model comprising at least one attention model and a prediction model, the prediction model being used to output the evaluation information based on the aggregate feature representation.

In some implementations, determining the first group of inputs comprises: using a multi-head attention mechanism to update the first feature representation based on edge features corresponding to an edge and nodes features of a pair of nodes corresponding to the edge in the first feature representation; and determining the first group of inputs based on the updated first feature representation.

In some implementations, the complex structure is a target complex structure, and the method further comprises: determining a binding pocket of the protein molecule; determining a first group of candidate complex structures based on the binding pocket of the protein molecule; and determining the target complex structure from the group of candidate complex structures based on binding free energy corresponding to the first group of candidate complex structures, wherein determining evaluation information on the binding between the ligand molecule and the protein molecule comprises: determining the effectiveness of the binding of the ligand molecule and the protein molecule.

In some implementations, the ligand molecule is a ligand molecule in the group of candidate ligand molecules, and the method further comprises: determining a first group of evaluation information that indicates the binding effectiveness between the protein molecule and the group of candidate ligand molecules; and determining a target ligand molecule from the group of candidate ligand molecules based on the binding effectiveness indicated by the first group of evaluation information.

In some implementations, the complex structure is a candidate complex structure in a second group of candidate complex structures, the second group of candidate complex structures corresponding to a group of candidate binding poses, and the method further comprises: determining a second group of evaluation information corresponding to the second group of candidate complex structures, the second group of evaluation information indicating the affinity of the group of candidate binding poses; and determining a target binding pose from the group of candidate binding poses based on the second group of evaluation information.

In a second aspect, a device is provided. The device comprises: a processing unit; and a memory coupled to the processing unit and comprising instructions stored thereon which, when executed by the processing unit, cause the device to perform acts comprising: obtaining a first feature representation of a ligand molecule and a second feature representation of a protein molecule, the first feature representation being determined based on a structure of the ligand molecule, the second feature representation being determined based on a structure of the protein molecule; determining a third feature representation of a complex structure, the complex structure being built based on the protein molecule and the ligand molecule; generating an aggregate feature representation based on the first feature representation, the second feature representation and the third feature representation; and determining evaluation information (which may also be referred to as prediction information) on the binding between the ligand molecule and the protein molecule based on the aggregate feature representation, the evaluation information indicating the effectiveness of the binding or indicating the affinity of a binding pose of the binding.

In some implementations, at least one of the first feature representation, the second feature representation and the third feature representation is a feature graph comprising node features and edge features, the node features being used to characterize atoms in a molecule, the edge features being used to characterize binding relationships between atoms.

In some implementations, generating an aggregate feature representation based on the first feature representation, the second feature representation and the third feature representation comprises: determining a first group of inputs based on the first feature representation, the second feature representation and the third feature representation; using an attention model to process the first group of inputs so as to determine a first intermediate feature representation; and determining the aggregate feature representation based on the first intermediate feature representation.

In some implementations, the attention model comprises a first attention model, and determining the aggregate feature representation based on the first intermediate feature representation comprises: determining a second group of inputs based on the first feature representation, the second feature representation and the first intermediate feature representation; using a second attention model to process the second group of inputs so as to determine a second intermediate feature representation; and determining the aggregate feature representation based on the second intermediate feature representation.

In some implementations, a parameter of the attention model is determined based on training a binding analysis model, the binding analysis model comprising at least one attention model and a prediction model, the prediction model being used to output the evaluation information based on the aggregate feature representation.

In some implementations, determining the first group of inputs comprises: using a multi-head attention mechanism to update the first feature representation based on edge features corresponding to an edge and nodes features of a pair of nodes corresponding to the edge in the first feature representation; and determining the first group of inputs based on the updated first feature representation.

In some implementations, the complex structure is a target complex structure, and the acts further comprise: determining a binding pocket of the protein molecule; determining a first group of candidate complex structures based on the binding pocket of the protein molecule; and determining the target complex structure from the group of candidate complex structures based on binding free energy corresponding to the first group of candidate complex structures, wherein determining evaluation information on the binding between the ligand molecule and the protein molecule comprises: determining the effectiveness of the binding of the ligand molecule and the protein molecule.

In some implementations, the ligand molecule is a ligand molecule in the group of candidate ligand molecules, and the acts further comprise: determining a first group of evaluation information that indicates the binding effectiveness between the protein molecule and the group of candidate ligand molecules; and determining a target ligand molecule from the group of candidate ligand molecules based on the binding effectiveness indicated by the first group of evaluation information.

In some implementations, the complex structure is a candidate complex structure in a second group of candidate complex structures, the second group of candidate complex structures corresponding to a group of candidate binding poses, and the acts further comprise: determining a second group of evaluation information corresponding to the second group of candidate complex structures, the second group of evaluation information indicating the affinity of the group of candidate binding poses; and determining a target binding pose from the group of candidate binding poses based on the second group of evaluation information.

In a third aspect, a computer program product is provided. The computer program product is tangibly stored in a non-transitory computer storage medium and comprises machine-executable instructions which, when executed by a device, causing the device to perform acts comprising: obtaining a first feature representation of a ligand molecule and a second feature representation of a protein molecule, the first feature representation being determined based on a structure of the ligand molecule, the second feature representation being determined based on a structure of the protein molecule; determining a third feature representation of a complex structure, the complex structure being built based on the protein molecule and the ligand molecule; generating an aggregate feature representation based on the first feature representation, the second feature representation and the third feature representation; and determining evaluation information (which may also be referred to as prediction information) on the binding between the ligand molecule and the protein molecule based on the aggregate feature representation, the evaluation information indicating the effectiveness of the binding or indicating the affinity of a binding pose of the binding.

In some implementations, at least one of the first feature representation, the second feature representation and the third feature representation is a feature graph comprising node features and edge features, the node features being used to characterize atoms in a molecule, the edge features being used to characterize binding relationships between atoms.

In some implementations, generating an aggregate feature representation based on the first feature representation, the second feature representation and the third feature representation comprises: determining a first group of inputs based on the first feature representation, the second feature representation and the third feature representation; using an attention model to process the first group of inputs so as to determine a first intermediate feature representation; and determining the aggregate feature representation based on the first intermediate feature representation.

In some implementations, the attention model comprises a first attention model, and determining the aggregate feature representation based on the first intermediate feature representation comprises: determining a second group of inputs based on the first feature representation, the second feature representation and the first intermediate feature representation; using a second attention model to process the second group of inputs so as to determine a second intermediate feature representation; and determining the aggregate feature representation based on the second intermediate feature representation.

In some implementations, a parameter of the attention model is determined based on training a binding analysis model, the binding analysis model comprising at least one attention model and a prediction model, the prediction model being used to output the evaluation information based on the aggregate feature representation.

In some implementations, determining the first group of inputs comprises: using a multi-head attention mechanism to update the first feature representation based on edge features corresponding to an edge and nodes features of a pair of nodes corresponding to the edge in the first feature representation; and determining the first group of inputs based on the updated first feature representation.

In some implementations, the complex structure is a target complex structure, and the acts further comprise: determining a binding pocket of the protein molecule; determining a first group of candidate complex structures based on the binding pocket of the protein molecule; and determining the target complex structure from the group of candidate complex structures based on binding free energy corresponding to the first group of candidate complex structures, wherein determining evaluation information on the binding between the ligand molecule and the protein molecule comprises: determining the effectiveness of the binding of the ligand molecule and the protein molecule.

In some implementations, the ligand molecule is a ligand molecule in the group of candidate ligand molecules, and the acts further comprise: determining a first group of evaluation information that indicates the binding effectiveness between the protein molecule and the group of candidate ligand molecules; and determining a target ligand molecule from the group of candidate ligand molecules based on the binding effectiveness indicated by the first group of evaluation information.

In some implementations, the complex structure is a candidate complex structure in a second group of candidate complex structures, the second group of candidate complex structures corresponding to a group of candidate binding poses, and the acts further comprise: determining a second group of evaluation information corresponding to the second group of candidate complex structures, the second group of evaluation information indicating the affinity of the group of candidate binding poses; and determining a target binding pose from the group of candidate binding poses based on the second group of evaluation information.

The functionality described herein can be performed, at least in part, by one or more hardware logic components. For example, and without limitation, illustrative types of hardware logic components that can be used include Field-Programmable Gate Arrays (FPGAs), Application-specific Integrated Circuits (ASICs), Application-specific Standard Products (ASSPs). System-on-a-chip systems (SOCs), Complex Programmable Logic Devices (CPLDs), and the like

Program code for carrying out methods of the subject matter described herein may be written in any combination of one or more programming languages. These program codes may be provided to a processor or controller of a general purpose computer, a special purpose computer, or other programmable data processing apparatus, such that the program codes, when executed by the processor or controller, cause the functions/operations specified in the flowcharts and/or block diagrams to be implemented. The program code may execute entirely on a machine, partly on the machine, as a stand-alone software package, partly on the machine and partly on a remote machine or entirely on the remote machine or a server.

In the context of this subject matter described herein, a machine-readable medium may be any tangible medium that may contain, or store a program for use by or in connection with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of the machine-readable storage medium would include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Further, although operations are depicted in a particular order, it should be understood that the operations are required to be executed in the particular order shown or in a sequential order, or all operations shown are required to be executed to achieve the expected results. In certain circumstances, multitasking and parallel processing may be advantageous. Likewise, while several specific implementation details are contained in the above discussions, these should not be construed as limitations on the scope of the subject matter described herein. Certain features that are described in the context of separate implementations may also be implemented in combination in a single implementation. Conversely, various features that are described in the context of a single implementation may also be implemented in multiple implementations separately or in any suitable sub-combination.

Although the subject matter has been described in language specific to structural features and/or methodological acts, it is to be understood that the subject matter specified in the appended claims is not necessarily limited to the specific features or acts described above. Rather, the specific features and acts described above are disclosed as example forms of implementing the claims.

1. A method for molecular binding analysis, comprising:
obtaining a first feature representation of a ligand molecule and a second feature representation of a protein molecule, the first feature representation being determined based on a structure of the ligand molecule, the second feature representation being determined based on a structure of the protein molecule;
determining a third feature representation of a complex structure, the complex structure being built based on the protein molecule and the ligand molecule;
generating an aggregate feature representation based on the first feature representation, the second feature representation and the third feature representation; and
determining evaluation information on the binding between the ligand molecule and the protein molecule based on the aggregate feature representation, the evaluation information indicating the effectiveness of the binding or indicating the affinity of a binding pose of the binding.

2. The method of claim 1, wherein at least one of the first feature representation, the second feature representation and the third feature representation is a feature graph comprising node features and edge features, the node features being used to characterize atoms in a molecule, the edge features being used to characterize binding relationships between atoms.

3. The method of claim 2, wherein generating an aggregate feature representation based on the first feature representation, the second feature representation and the third feature representation comprises:
determining a first group of inputs based on the first feature representation, the second feature representation and the third feature representation;
using an attention model to process the first group of inputs so as to determine a first intermediate feature representation; and
determining the aggregate feature representation based on the first intermediate feature representation.

4. The method of claim 3, wherein the attention model comprises a first attention model, and determining the aggregate feature representation based on the first intermediate feature representation comprises:
determining a second group of inputs based on the first feature representation, the second feature representation and the first intermediate feature representation;
using a second attention model to process the second group of inputs so as to determine a second intermediate feature representation; and
determining the aggregate feature representation based on the second intermediate feature representation.

5. The method of claim 3, wherein a parameter of the attention model is determined based on training a binding analysis model, the binding analysis model comprising at least one attention model and a prediction model, the prediction model being used to output the evaluation information based on the aggregate feature representation.

6. The method of claim 3, wherein determining the first group of inputs comprises:
using a multi-head attention mechanism to update the first feature representation based on edge features corresponding to an edge and nodes features of a pair of nodes corresponding to the edge in the first feature representation; and
determining the first group of inputs based on the updated first feature representation.

7. The method of claim 1, wherein the complex structure is a target complex structure, and the method further comprises:
determining a binding pocket of the protein molecule;
determining a first group of candidate complex structures based on the binding pocket of the protein molecule; and
determining the target complex structure from the group of candidate complex structures based on binding free energy corresponding to the first group of candidate complex structures,
wherein determining evaluation information on the binding between the ligand molecule and the protein molecule comprises:
determining the effectiveness of the binding of the ligand molecule and the protein molecule.

8. The method of claim 1, wherein the ligand molecule is a ligand molecule in the group of candidate ligand molecules, and the method further comprises:
determining a first group of evaluation information that indicates the binding effectiveness between the protein molecule and the group of candidate ligand molecules; and
determining a target ligand molecule from the group of candidate ligand molecules based on the binding effectiveness indicated by the first group of evaluation information.

9. The method of claim 1, wherein the complex structure is a candidate complex structure in a second group of candidate complex structures, the second group of candidate complex structures corresponding to a group of candidate binding poses, and the method further comprises:
determining a second group of evaluation information corresponding to the second group of candidate complex structures, the second group of evaluation information indicating the affinity of the group of candidate binding poses; and
determining a target binding pose from the group of candidate binding poses based on the second group of evaluation information.

10. A device, comprising:
a processing unit; and
a memory coupled to the processing unit and comprising instructions stored thereon which, when executed by the processing unit, cause the device to implement the method of any of claims 1 to 9.

11. A computer program product, being tangibly stored in a computer storage medium and comprising machine-executable instructions which, when executed by a device, causing the device to implement the method of any of claim 1.
